(19) United States
(12) Patent Application Publication
(10) Pub. No.: US 2017/0014417 A1
(43) Pub. Date: Jan. 19, 2017

(54) PHARMACEUTICAL ADMINISTRATION SYSTEM FOR THE TRANSDERMAL APPLICATION OF VARDENAFIL

(71) Applicant: LIPP LIFE SCIENCES LLC, Zionsville, IN (US)

(72) Inventor: Ralph LIPP, Zionsville, IN (US)

(73) Assignee: LIPP LIFE SCIENCES LLC, Zionsville, IN (US)

(21) Appl. No.: 14/799,454

(22) Filed: Jul. 14, 2015

(51) Int. Cl. A61K 31/53; A61K 9/70; A61K 31/568

(52) U.S. Cl. CPC A61K 31/53; A61K 31/568; A61K 9/7038

(57) ABSTRACT

A pharmaceutical administration system for the transdermal application of at least one active agent includes vardenafil and/or a pharmaceutically acceptable salt thereof as the active agent and a pharmaceutically acceptable carrier providing a solution of the at least one active agent in the administration system. Another active agent may be testosterone. This system can be used to systemically deliver therapeutic doses of vardenafil and/or a pharmaceutically acceptable salt thereof and optionally testosterone in order to treat benign prostatic hyperplasia, erectile dysfunction, male hypogonadism, pulmonary hypertension, and/or pulmonary arterial hypertension.

### BACKGROUND OF THE INVENTION

1. Field of the Invention

The present invention relates to a pharmaceutical administration system for transdermal delivery and a use of the system. In particular, the system comprises vardenafil and/or a pharmaceutically acceptable salt thereof as an active agent and optionally testosterone as a second active agent.

2. Description of the Background Art

Vardenafil is a phosphodiesterase type 5 (PDE V) inhibitor, which is used in oral immediate release formulations for the on-demand treatment of erectile dysfunction (ED). It has an absolute bioavailability of approximately 15% upon oral dosing in man. Due to its short biological half-life, which does not allow for continuously elevated plasma levels of the drug upon once daily oral dosing, vardenafil (and/or a pharmaceutically acceptable salt thereof) has not been introduced into important therapeutic indications for PDE V inhibitors such as pulmonary hypertension (PH), pulmonary arterial hypertension (PAH), continuous treatment of ED, and benign prostatic hyperplasia (BPH), among others. Furthermore, no transdermal products containing vardenafil are on the market.

As such, there is currently a need for pharmaceutical administration systems that deliver continuous plasma levels of vardenafil, preferably in a non-invasive, patient-accepted, and easy to adhere way.

Testosterone is being used in the treatment of androgen deficiency and male hypogonadism in various formulations, including transdermal gels and patches. However, no oral products containing testosterone are currently on the market. Marketed intramuscular formulations and an oral product are based on non-physiological esters of testosterone. Patients suffering from male hypogonadism and erectile dysfunction benefit from receiving both continuous delivery of testosterone through intramuscular application of a testosterone ester as well as oral on-demand application of vardenafil tablets. However, the drawbacks of this combination include the invasiveness of the intramuscular injection, the need for a health care professional to administer the product (i.e., not patient self-administered), the use of non-physiological testosterone esters, and the need of following a second regimen by taking the PDE V inhibitor via the oral route on demand as well.

As such, there is currently a need for patient-administered and non-invasive pharmaceutical systems and regimens for a continuously combined delivery of the physiological hormone testosterone in combination with vardenafil and/or a pharmaceutically acceptable salt thereof.

Out of 2,500 medical entities approved by the FDA, only 23 medical entities (e.g., estradiol, levonorgestrel, and nicotine) were approved for use via a transdermal patch by the FDA in 2013. This number is limited because the skin provides a significant barrier, and many of the approved medical entities require too high daily doses and/or do not possess physicochemical characteristics that allow for sufficiently high transdermal flux rates. Moreover, marketed combination transdermal systems that deliver two active agents simultaneously are even fewer, and they typically deliver active agents from the same structural class such as sex steroids (e.g., estradiol in combination with a progestin like levonorgestrel or gestodene).

More specifically, transdermal preparations are well accepted by patients for a number of different therapeutic indications mainly due to their non-invasiveness, the ease-of-use of transdermal products, and the relatively long dosing intervals that can be achieved even for drugs with a short biological half-life. Despite the significant scientific and technological efforts in the area of transdermal drug delivery since U.S. Pat. No. 3,598,122 was granted in 1971, only 19 medical entities had obtained FDA approval by 2007 in the form of transdermal patches and delivery systems (Prausnitz M R, Langer R., Transdermal drug delivery. Nat Biotechnol. 2008; 26(11):1261-1268.) and 23 had obtained approval by 2013 (R. Lipp, Status quo, challenges and opportunities of transdermal drug delivery, Am. Pharmaceut. Rev. 17 (2014) 22-28). The low number of medical entities successfully incorporated in marketed transdermal patches for human use out of the approximately 2500 medical entities approved by the FDA (Huang R, Southall N, Wang Y, et al. The NCGC Pharmaceutical Collection: A comprehensive resource of clinically approved drugs enabling repurposing and chemical genomics. Sci Transl Med. 2011; 3(80):16.) can be explained by the fact that the human skin provides an excellent barrier for most approved medical entities. Furthermore, it is even more challenging to combine two different medical entities for simultaneous transdermal delivery from one and the same dose form. Looking at marketed transdermal patches and delivery systems shows that the few available combination products typically deliver medical entities, which belong to the same structural class or to structurally related classes. By 2007, marketed combination systems in the U.S. comprised transdermal patches based on combinations of sex hormones: Combipatch, containing estradiol and norethidrone, Ortho Evra®, containing ethinyl estradiol and norelgestromin, and Climara® Pro, containing estradiol and levonorgestrel. The fact that there are so few transdermal combination patches available to patients and no transdermal combination patches that contain medical entities from different structural classes can be explained by the fact that the simultaneous transdermal delivery of two physico-chemically different medical entities through human skin at therapeutically relevant flux rates is very difficult to achieve.

The phosphodiesterase-5 inhibitor vardenafil is being used under the trade name Levitra® in tablets for peroral administration and under the trade name Staxyn® in orally disintegrating tablets. The indication of both products is erectile dysfunction. Both formulations are to be taken on demand approximately 60 minutes before sexual activity. Vardenafil is rapidly absorbed upon oral administration with a median time to reach its maximum concentration in plasma of 1.5 hours after administration in the fasted state (Staxyn® prescribing information). Vardenafil has a plasma half-life of approximately 3 h after oral administration, which is too short for oral once-a-day administration of the aforementioned products in indications which require an elevated plasma concentration of the active agent for an extended period of time. In order to extend the use of vardenafil beyond the on demand treatment of erectile dysfunction, formulations and routes of delivery are needed that allow for more continuous plasma levels. In particular, patient-friendly formulations, which can be self-administered by the patient and provide longer dosing intervals like once-a-day, twice-a-week, and once-a-week are desirable.

Based on the physicochemical properties of vardenafil (e.g., molecular weight, salt form, and log P) and the required daily doses of the drug to treat patients suffering from PH, PAH, BPH, and ED, the ability to create sufficiently high transdermal flux rates to enable a transdermal product seemed highly unlikely. Furthermore, given vardenafil's significant structural differences from testosterone (e.g., molecular weight, charge, and lipophilicity), the feasibility of a transdermal combination product delivering both vardenafil and testosterone at the required flux rates seemed extremely unlikely. Specifically, vardenafil has a molecular weight of 488.60, and testosterone has a molecular weight of 288.42. Vardenafil has a log P value of 1.4, and testosterone has a log P value of 3.4. Finally, vardenafil is basic, and testosterone is neutral.

### SUMMARY OF THE INVENTION

Surprisingly, the present invention finds that vardenafil and/or a pharmaceutically acceptable salt thereof can be delivered via the human skin at significant flux rates. Furthermore, and also surprisingly, vardenafil (and/or a pharmaceutically acceptable salt thereof) and testosterone can be simultaneously delivered through the human skin with a single formulation at significant flux rates. Thus, the present invention allows patients to self-administer the drug from transdermal compositions in a non-invasive and highly accepted way at extended dosing intervals of, for example, once-a-day, twice-a-week, or once-a-week. These transdermal compositions therefore enable the therapeutic use of vardenafil in indications other than on demand treatment of erectile dysfunction in a highly patient accepted way. Such indications include continuous treatment of erectile dysfunction, treatment of benign prostatic hyperplasia, and treatment of peripheral arterial hypertension, among others.

Yasin et al. showed (Yassin D J, Yassin A A, Hammerer P G, Combined testosterone and vardenafil treatment for restoring erectile function in hypogonadal patients who failed to respond to testosterone therapy alone. J Sex Med. 2014 February; 11(2):543-52) that the use of oral vardenafil on demand as an adjunctive therapy to intramuscular injections of testosterone undecanoate provided in 3 month intervals improved the scores on the International Index of Erectile Function Questionnaire-five items in testosterone deficient men who did not respond to testosterone undecanoate alone. However, the regimen patients used in the course of this study is complicated in that they had to combine the oral application of vardenafil on demand with intramuscular injections of a depot formulation of testosterone undecanoate every three months. This regimen is invasive and thus painful in nature. The intramuscular injections, also, cannot be self-administered by the patient. Furthermore, this combination is complex and thus not very attractive to patients.

The use of transdermal mono formulations of testosterone as a way of treating androgen deficiency in men has been previously demonstrated in U.S. Pat. Nos. 6,503,894 B1, 8,486,925 B2, 8,435,944 B2, 8,178,518 B2, and 6,579,865 B2. There are no oral products containing testosterone on the market. Furthermore, U.S. 2005/0049233 A1 describes the use of combining the transdermal once-a-day application of testosterone (Androgel®, 1%) with the on demand oral application of the phosphodiesterase-5 inhibitor sildenafil (Viagra®) as a method for improving sexual performance in Sildenafil® non-responders. The patient need for a non-invasive simultaneous combination formulation of a phosphodiesterase-5 inhibitor with an androgen allowing for a simultaneous administration of both from one dose form, however, is not addressed with this regimen.

U.S. 2005/0049233 A1 describes in Example 1 that the application of 5 g per day of the testosterone gel Androgel® (1%) to the skin plus 100 mg of oral sildenafil (1 hour before intercourse) improved sexual performance in sildenafil non-responders. However, it has been concluded by Spitzer et al. that “Sildenafil plus testosterone was not superior to sildenafil plus placebo in improving erectile function in men with erectile dysfunction and low testosterone levels” (Spitzer et al., “Effect of Testosterone Replacement on Response to Sildenafil Citrate in Men With Erectile Dysfunction: A Parallel, Randomized Trial.” Ann Intern Med. 2012; 157, pages 681-691). Therefore, combining oral sildenafil therapy with transdermal testosterone therapy in patients suffering from erectile dysfunction yields inconsistent and unpredictable results.

Spitzer et al. treated patients in a sildenafil monotherapy dose-optimization phase for 3 to 7 weeks, resulting in an optimized dose for the individual patient of either 25 mg, 50 mg or 100 mg sildenafil orally on demand. Thereafter, 70 of these patients were assigned to receive the optimized dose of orally administered sildenafil in combination with a placebo gel, whereas a second group of 70 patients was assigned to continue their optimized dose of oral sildenafil but combined with 10 g of 1% transdermal testosterone gel (Testim®) applied daily to their skin for 2 weeks. After 2 weeks, the testosterone level of the subjects in the second group was measured, and, if deemed appropriate, the dose of transdermal testosterone gel was adjusted to either 5 g per day or 15 g per day. Spitzer et al. found, “In men with ED who had low testosterone levels, the addition of a replacement dose of testosterone to an optimized dose of sildenafil was not associated with greater improvement in erectile function than that associated with addition of placebo gel. Sildenafil plus testosterone was not superior to sildenafil plus placebo in improving any domain of sexual function, frequency of total or satisfactory sexual encounters, vitality, ED-related quality of life, or marital intimacy.”

It has now been found that surprisingly vardenafil and testosterone can be simultaneously delivered through human skin at therapeutically relevant flux rates from transdermal combination formulations containing both medical entities. This has been achieved despite the fact that both medical entities stem from different structural classes and have significantly different physico-chemical properties. Vardenafil belongs to the class of phosphodiesterase-5 inhibitors and is a basic molecule with a relative mass of 488.6 and a log P of 1.4. In contrast, testosterone belongs to the class of sex steroids and is a neutral molecule with a relative mass of 288.4 and a log P of 3.4. The combination formulations according to this invention allow the simultaneous transdermal delivery of both medical entities to treat various therapeutic indications including erectile dysfunction in androgen deficient men who do not respond to transdermal testosterone alone, male hypogonadism, low testosterone levels, erectile dysfunction, and male disorders, among others.

In a first embodiment, the present invention is directed to a pharmaceutical administration system for the transdermal application of at least one active agent comprising vardenafil and/or a pharmaceutically acceptable salt thereof as the active agent and a pharmaceutically acceptable carrier providing a solution, partial solution, or dispersion of the at least one active agent in the administration system. The system may also comprise testosterone as another active agent. A content of vardenafil and/or a pharmaceutically acceptable salt thereof in the system may be 0.1-15% by weight, and a content of testosterone in the system may be 0.1-15% by weight. The pharmaceutically acceptable carrier may be an alcohol or a mixture of alcohols, or the pharmaceutically acceptable carrier may comprise a mixture of one or more alcohols and water. The pharmaceutically acceptable carrier may also be a biocompatible pressure sensitive adhesive or a mixture of biocompatible pressure sensitive adhesives. The system may further comprise a gel forming agent, a transdermal penetration enhancer, a second transdermal penetration enhancer, and/or a crystallization inhibitor.

In a second embodiment, the present invention is directed to a method of systemically delivering therapeutic doses of vardenafil and/or a pharmaceutically acceptable salt thereof, and optionally testosterone, comprising applying the system described above to the skin of a patient in need thereof. The method may be used to treat benign prostatic hyperplasia, erectile dysfunction, male hypogonadism, pulmonary hypertension (PH), and/or pulmonary arterial hypertension.

Further scope of the applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to one of ordinary skill in the art from this detailed description.

### DETAILED DESCRIPTION OF THE INVENTION

The present invention will now be described.

The present invention is directed to a pharmaceutical administration system.

One embodiment of a pharmaceutical administration system of the invention for the transdermal application of at least one active agent will now be described. The first active agent is a PDE V inhibitor and/or a pharmaceutically acceptable salt thereof. The PDE V inhibitors may include, but are not limited to, vardenafil and/or pharmaceutically acceptable salts thereof. A preferred example of a PDE V inhibitor is vardenafil and/or a pharmaceutically acceptable salt thereof. The system also includes a pharmaceutically acceptable carrier providing a solution of the at least one active agent in the administration system.

Vardenafil can be used as a free base or as one of its pharmaceutically acceptable salts, including, among others, vardenafil hydrochloride, to create the transdermal compositions described in the present invention. The transdermal compositions containing vardenafil alone or in combination with medical entities from the groups of androgens, antiestrogens, etc. include the transdermal systems known to one of ordinary skill in the art and include liquid formulations, sprays, gels, transdermal matrix patches, transdermal reservoir patches, creams, ointments, and emulsions. The transdermal formulations are applied to the skin of the patient to deliver the medical entities they contain transdermally over an extended period of time.

A second active agent may be an androgen. Preferred examples of androgens are testosterone and derivatives of testosterone such as esters of testosterone. However, vardenafil may be the only active agent, and other active agents may be excluded.

As used herein, the term “active agent” means a compound that, when administered to a patient, confers, directly or indirectly, a physiological effect on the patient. For the present invention, a physiological effect would involve treating conditions like pulmonary hypertension (PH), pulmonary arterial hypertension (PAH), benign prostatic hyperplasia (BPH), erectile dysfunction (ED), male hypogonadism, and male disorders, among others. Examples of active agents in the context of the present invention are vardenafil and androgens.

In addition, combinations of other active agents with vardenafil and/or a pharmaceutically acceptable salt thereof are also possible. For example, other active agents may include antiestrogens such as clomifene, enclomifene, and fispemifene; prostacyclin and synthetic analogs thereof such as iloprost, treprostinil, and epoprostenol; endothelin receptor antagonists such as ambrisentan and macicentan; and soluble guanylate cyclase stimulators such as riociguat.

For ED, BPH, testosterone deficiency, and other male disorders, the second active agent may be testosterone, testosterone cypionate, testosterone enanthate, testosterone undecanoate, testosterone propionate, pro-drugs of testosterone in general, methyltestosterone, androgens in general, clomifene, enclomifene, fispemifene, antiestrogens in general, pharmaceutically acceptable salts thereof, and mixtures thereof.

For PH, PAH, and other cardiovascular diseases, the second active agent may be a prostacyclin and a synthetic derivative and analog of prostacyclins, in particular iloprost and its beta-cyclodextrine clathrate, treprostinil, epoprostenol, and beraprost; endothelin receptor antagonists, in particular ambrisentan and macicentan; soluble guanylate cyclase stimulators, in particular riociguat; pharmaceutically acceptable salts thereof; and mixtures thereof.

A content of the PDE V inhibitor (preferably vardenafil and/or a pharmaceutically acceptable salt thereof) in the system may be 0.1-15% by weight, preferably 0.2 to 10% by weight, and most preferably 0.3 to 8% by weight.

A content of the androgen (preferably testosterone) in the system may be 0.1-15% by weight, preferably 0.2 to 10% by weight, and most preferably 0.3 to 8% by weight.

The pharmaceutically acceptable carrier may be any pharmaceutically acceptable carrier that provides a solution of the at least one active agent in the administration system. For example, the pharmaceutically acceptable carrier may be an alcohol or a mixture of alcohols. As another example, the pharmaceutically acceptable carrier may comprise a mixture of one or more alcohols and water. As yet another example, the pharmaceutically acceptable carrier is a biocompatible pressure sensitive adhesive or a mixture of biocompatible pressure sensitive adhesives. Another general type of carrier would be an emulsion system based on a hydrophilic phase and a hydrophobic phase.

The system may also include other additives. The additives may include penetration enhancers, solvents and co-solvents, gel forming agents and thickening agents, bases, crystallization inhibitors, adhesives, backing materials, release rate controlling membranes, release liners, and antioxidants. Preferred additives include a gel forming agent, a transdermal penetration enhancer, a second transdermal penetration enhancer, a crystallization inhibitor, and an antioxidant. However, any of these additives may also be excluded. A content of the transdermal penetration enhancer in the system may be 0.1-25% by weight, preferably 0.2-15% by weight, and most preferably 0.5-10% by weight. A content of the crystallization inhibitor in the system may be 0.1-25% by weight, preferably 0.5-20% by weight, most preferably 1-15% by weight. A content of the antioxidant in the system may be 0.01-5% by weight, preferably 0.05-3% by weight, and most preferably 0.1-2% by weight.

Examples of a gel forming agent include polyacrylic acids, cellulose derivatives, organic polymers, and mixtures thereof. The polyacrylic acids may be carbomer 940, carbomer 980, carbomer 981, carbopol Ultrez 10, carbopol 934P, and mixtures thereof. The polyacrylic acid is preferably carbomer 980. The cellulose derivatives may be methyl cellulose, hydroxypropyl cellulose, hydroxypropylmethyl cellulose, ethyl cellulose, carboxy methyl cellulose, and mixtures thereof. The cellulose derivative is preferably hydroxypropyl cellulose, for example Klucel® MF.

Examples of a transdermal penetration enhancer include organic amines, fatty acids, esters, lipophilic alcohols, pyrrolidones, and mixtures thereof. The system of the present invention may have one or more transdermal penetration enhancers. Preferably, the system of the present invention has one transdermal penetration enhancer. The organic amines may be lauryl amine, myristyl amine, bis(2-hydroxypropyl)amine, and mixtures thereof. The organic amine is preferably bis(2-hydroxypropyl)amine. The fatty acids may be lauric acid, myristic acid, caprylic acid, oleic acid, and mixtures thereof. The fatty acid is preferably myristic acid. The esters may be isopropyl myristate, ethyl oleate, oleyl acetate, and mixtures thereof. The ester is preferably isopropyl myristate. The lipophilic alcohols may be lauryl alcohol, myristyl alcohol, cetyl alcohol, octyl alcohol, and mixtures thereof. The lipophilic alcohol is preferably lauryl alcohol. The pyrrollidones may be 2-pyrrollidone, N-methyl-2-pyrrolidone, and mixtures thereof. The pyrrollidone is preferably N-methyl-2-pyrrolidone.

The solvents and co-solvents may form a solvent system. Examples of solvents include water, water soluble alcohols, polyols, and mixtures thereof. The water soluble alcohols may include ethanol, propanol, isopropanol, and mixtures thereof. The water soluble alcohol is preferably ethanol. The polyols may be 1,2-propane diol, polyethylene glycol, glycerol, and mixtures thereof. The polyol is preferably 1,2-propane diol.

The bases are for activating polyacrylic acids and for gel formation as well as for setting the base of vardenafil free in situ when working with a pharmaceutically acceptable salt of vardenafil (e.g., vardenafil hydrochloride) instead of vardenafil. The bases may include inorganic bases, organic bases, and mixtures thereof. The inorganic basis may include sodium hydroxide, 5 N aqueous sodium hydroxide solution, 1 N aqueous sodium hydroxide solution, 0.1 N aqueous solution, potassium hydroxide, ammonium hydroxide, and mixtures thereof. The inorganic base is preferably sodium hydroxide. The organic bases may include bis(2-hydroxypropyl)amine, tri(2-hydroxypropyl)amine, triethanolamine, L-arginine, aminomethylpropanol, and mixtures thereof. The organic base is preferably bis(2-hydroxypropyl)amine.

Examples of a crystallization inhibitor include organic polymers, silicone dioxide and derivatives thereof, and mixtures thereof. The organic polymers may include polyvinyl pyrrolidone (e.g., BASF's Kollidon® 12 PF, Kollidon® 17 PF, Kollidon® 25, Kollidon® 30, and Kollidon® 90 F), vinylpyrrolidone vinyl acetate copolymer (copovidone, e.g., Kollidon® VA 64), Soluplus®, and mixtures thereof. The organic polymer is preferably Kollidon® VA 64. The silicone dioxide and derivatives thereof may include colloidal silicon dioxide (e.g., Aerosil® 200, Aerosil® 200 VV, Aerosil® 300 Pharma, Aeroperl® 300 Pharma), hydrophobic silicon dioxides (e.g., Aerosil® 972 Pharma), and mixtures thereof. The silicon dioxide and derivatives thereof are preferably Aerosil® 200 colloidal silicon dioxide.

The adhesives include all classes of skin tolerated adhesives. Examples of the adhesives include polyacrylate adhesives, polyisobutylene adhesives, silicone adhesives, and mixtures thereof. The polyacrylate adhesives may include Henkel's DuroTak® 87-4098, DuroTak® 387-2287, DuroTak® 387-4287, DuroTak® 387-2516, DuroTak® 87-900A, DuroTak® 87-9301, DuroTak® 387-2510, DuroTak® 87-2074, DuroTak® 87-235A, DuroTak® 387-2353, DuroTak® 387-2052, DuroTak® 387-2852, DuroTak® 387-2051, DuroTak® 387-2054, DuroTak® 387-2194, DuroTak® 387-2196, Gelva® GMS 788, Gelva® GMS 9073, Gelva® GMS 3083, Gelva® GMS 2723, and Gelva® GMS 3253; Evonik's Eudragit® E 100; a mixture of Eudragit® E 100 with 5-35% acetyltributyl citrate and/or 1-10% succinic acid; Adhesives Research's ARcare® A-4984, ARcare® A-4753, ARcare® A-5149, ARcare® A-5095, and ARcare® A-3163; and mixtures thereof. The polyacrylate adhesive is preferably DuroTak® 87-4098. The polyisobutylene adhesives may include Henkel's DuroTak® 87-6908; Adhesives Research's ARcare® A-4607; and mixtures thereof. The polyisobutylene adhesive is preferably DuroTak® 87-4098. The silicone adhesives may include Dow Corning's Bio PSA 7-4401, Bio PSA 7-4402, Bio PSA 7-4501, Bio PSA 7-4502, Bio PSA 7-4601, Bio PSA 7-4602, Bio PSA 7-4101, Bio PSA 7-4102, Bio PSA 7-4201, Bio PSA 7-4202, Bio PSA 7-4301, Bio PSA 7-4302, Bio PSA 7-4560, and mixtures thereof. The silicone adhesive is preferably Bio PSA 7-4301.

Examples of the backing materials include mono layer films and multi-layer films. The mono layers films may include low density poly ethylene (LDPE), polyethylene (e.g., CoTran® 9722), ethylene-vinyl acetate (EVA), polyvinyl chloride (PVC), polyvinylidene chloride (PVDC, e.g., Saran® films), polyester, aliphatic polyamides (e.g., Nylon®), polyurethane (e.g., CoTran™ 9701), and polyethylene terephthalate. The mono layer films may be used alone or with the addition of fillers, plasticizers, pigments, and/or UV absorbers. The mono layer film is preferably polyvinylidene chloride (PVDC, e.g., Saran® films). The multi-layer films may include DOW™ BLF 2014, DOW™ BLF 2015, DOW™ BLF 2050, DOW™ BLF 2057, Scotchpak™ 9723, Scotchpak™ 9730, Scotchpak™ 9733, Scotchpak™ 9735, and Scotchpak™ 1012. The multi-layer films may be used alone or with the addition of fillers, plasticizers, pigments, and/or UV absorbers. The multi-layer film is preferably DOW™ BLF 2050.

Examples of the release rate controlling membranes include semipermeable polymer films. The semipermeable polymer films may include ethylene vinylacetate copolymer, microporous polypropylene, and polyethylene terephtalate/fluoropolymer. The semipermeable polymer film is preferably ethylene vinylacetate copolymer.

Examples of the release liners include siliconized liners and fluoropolymer coated liners. The siliconized liners may include Loparex Primeliner™, siliconized polyester, and siliconized PET. The siliconized liner is preferably siliconized polyester. The fluoropolymer coated liners may include Scotchpak™ 1022, Scotchpak™ 9741, and Scotchpak™ 9742. The liner is preferably Scotchpak™ 1022.

Examples of formulations of the present invention include 1% solutions of vardenafil (plus optionally 1% testosterone) in a solvent such as ethanol, 2-propanol, 1,2-propanediol with or without the addition of water. Mixed solvent systems (e.g., ethanol/2-propanol mixtures) may also be used. Another example includes a 3% solution of vardenafil in the matrix of a pressure sensitive adhesive (e.g., of the DuroTak® types) formulated as a monolithic transdermal patch with backing and a release liner.

Vardenafil (and/or a pharmaceutically acceptable salt thereof) is prone to oxidation in certain formulations. As such, the addition of antioxidants yields more stable transdermal compositions. See, for example, “Remington: The Science and Practice of Pharmacy,” D. B. Troy, P. Beringer (eds.), p. 747, Lippincott, Williams & Wilkins, Baltimore, Md., USA, 2005. Examples of the antioxidants include lipophilic antioxidants, hydrophilic antioxidants, and mixtures thereof. The lipophilic antioxidants may include, but are not limited to, butylated hydroxyanisole (BHA), butylated hydroxytoluene (BHT), alpha tocopherol, gallic acid, gallic acid esters, and mixtures thereof. The lipophilic antioxidant is preferably butylated hydroxyanisole (BHA). The hydrophilic antioxidants may include, but are not limited to, sodium thiosulfate, sodium ascorbate, ascorbic acid, and mixtures thereof. The hydrophilic antioxidant is preferably sodium ascorbate.

Patient acceptance of transdermal systems is dependent on system size. For transdermal patches, current marketed products range in size from below 5 cm² to over 40 cm². The present invention may have similar size ranges. In certain indications, multiple patches need to be applied. In general, patients prefer smaller patch sizes over larger ones and patches beyond a certain size (approximately 50 cm²) will not be practical or tolerated for a number of indications. For a given transdermal patch, there is typically a linear relationship between size and dose of the active/actives systemically delivered over the application period (e.g., 24 hours). Given the patient's preference for small systems, high and stable transdermal flux rates through human skin are objectives achieved by the transdermal patch of the present invention.

Specifically, the present invention achieved a cumulative flux rate for vardenafil through human skin over 24 hours of 0.1 μg/cm² or higher, preferably 0.3 μg/cm² or higher, and more preferably 1 μg/cm² or higher. In addition, an upper limit of 500 μg/cm² can be expected.

For testosterone, the present invention achieves a cumulative flux rate through human skin over 24 hours of 5 μg/cm² or higher, preferably 20 μg/cm² or higher, and more preferably 50 μg/cm² or higher. In addition, an upper limit of 1,000 μg/cm² can be expected.

Patient acceptance of liquid or semisolid transdermal formulations is dependent on the amount of formulation to be applied and consequently the area of skin to be covered. In general, smaller amounts of formulations are preferred over larger amounts. In testosterone replacement therapy, marketed formulations typically range from 1.25 g, to 5 g, to 10 g (or more) per single dose. The present invention may have similar ranges. Upon application of these formulations to the skin of the patient (e.g., arm, shoulder, torso, abdomen, etc.), typically an application area of 50 cm² to 200 cm² to 400 cm² (or more) will be covered with the formulation. For a given transdermal formulation, there is typically a linear relationship between amount formulation dosed and dose of the active/actives systemically delivered over the application period (e.g., 24 hours). Given the patient's preference for smaller amounts of formulation applied, high and stable transdermal flux rates through human skin are objectives achieved by the transdermal formulations of the present invention.

Specifically, for transdermal (non-patch) systems, including gels, liquids, sprays, emulsions and other liquid or semisolid types, the present invention achieved a cumulative flux rate for vardenafil through human skin over 24 hours of 1 μg/cm² or higher, preferably 5 μg/cm² or higher, and more preferably 20 μg/cm² or higher. In addition, an upper limit of 500 μg/cm² can be expected.

For testosterone, the present invention achieved a cumulative flux rate through human skin over 24 hours of 3 μg/cm² or higher, preferably 10 μg/cm² or higher, and more preferably 25 μg/cm² or higher. In addition, an upper limit of 1,000 μg/cm² can be expected.

Another aspect of the present invention relates to methods for delivering therapeutic doses of PDE V inhibitors (preferably vardenafil and/or a pharmaceutically acceptable salt thereof) and optionally androgens (preferably testosterone) and methods for treatment of certain diseases with the system of the invention. The preferred embodiments of the system in terms of the component of the system described above are also applicable to these aspects of the present invention.

This embodiment of the present invention relates to a method of systemically delivering therapeutic doses of PDE V inhibitors (preferably vardenafil and/or a pharmaceutically acceptable salt thereof) and optionally androgens (preferably testosterone) by applying the system described above to skin of a patient in need thereof. The method can be used for treating benign prostatic hyperplasia, erectile dysfunction, male hypogonadism, male hypogonadism and erectile dysfunction simultaneously, pulmonary hypertension (PH), and/or pulmonary arterial hypertension.

The system can be administered as a transdermal gel, lotion, emulsion, liquid, spray, patch, or other such formulations known for transdermal application.

Packaging and dosing information can be added as well.

The present invention will hereinafter be described with reference to exemplary embodiments, which are written to be understood only as examples and are not intended to limit the scope of the present application. The manufacturing processes described below can be scaled up to commercial scales of several hundred kilograms using manufacturing equipment and technologies known to one of ordinary skill in the art.

### EXAMPLES

### Example 1

### Formulation 1

In a 10 mL flask with a stopper, 100 mg of vardenafil (base; from LGM Pharma) and 400 mg of isopropylmyristate were mixed with 9.5 g of 2-propanol/water (70% v/v) and shaken for 10 minutes at ambient temperature to obtain the formulation below. The resulting crystal free formulation was subjected to an in vitro-permeation test through Heat Separated Human Epidermis (HSHE), which is highly predictive for in vivo-transdermal flux rates.

### Formulation 1

1% vardenafil
4% isopropylmyristate
95% 2-propanol/water (70% v/v)

Permeation experiments were conducted in duplicate (using 2 cells) following a Standard Operating Procedure for using Hanson Vertical Diffusion Cells (7 mL) mounted with Heat Separated Human Epidermis, an in vitro-experiment with high predictability for transdermal in vivo-fluxes.

Heat Separated Human Epidermis was prepared following the Standard Operating Procedure, in that the stratum corneum of the skin was protected from damage while subcutaneous fat tissue was cut off. Resulting skin samples were stored in airtight packaging between −20° C. and −30° C. Cellulose dialysis membrane was die cut into pieces of 25 mm in diameter and hydrated in distilled water for 24 h. Subsequently, the pieces of cellulose dialysis membrane were hydrated in the acceptor medium for the penetration experiment for one additional hour. From the frozen skin samples, specimen of 20 mm in diameter were die cut and thawed for 5 min. The 20 mm skin specimen were immersed in water of 60° C. for 90 seconds. Thereafter, the dermis was removed from the epidermis. After hydration in PBS buffer for 30 min the epidermis was applied onto hydrated cellulose dialysis membrane. The resulting Heat Separated Human Epidermis (HSHE) sheets were transferred into the Hanson Vertical Diffusion Cells and the permeation experiments were run at 32° C.

200 mg of the formulation were applied on each Heat Separated Human Epidermis. 0.5% Cavamax™ in PBS buffer served as the acceptor medium for the permeation experiments. Samples were withdrawn at predetermined time intervals (see Tables 1 and 2 below) and subjected to HPLC quantification of vardenafil (V).

TABLE 1
Results of Permeation Experiments with Formulation 1 for Cell 1

| Time | Cumulative amount permeated V |
|---|---|
| (h) | (μg/cm²) |
| 0 | 0.21138 |
| 2 | 1.95124 |
| 4 | 4.76908 |
| 8 | 7.60946 |
| 12 | 11.58070 |
| 16 | 15.57210 |
| 24 | 21.89370 |

TABLE 2
Results of Permeation Experiments with Formulation 1 for Cell 2

| Time | Cumulative amount permeated V |
|---|---|
| (h) | (μg/cm²) |
| 0 | Below lower limit of detection |
| 2 | 0.20782 |
| 4 | 0.53833 |
| 8 | 1.00294 |
| 12 | 2.00711 |
| 16 | 3.86842 |
| 24 | 14.73262 |

### Example 2

### Formulation 2

A 10% stock solution of Kollidon VA 64 was created by dissolving 10 g Kollidon VA 64 in 90 g 2-propanol under stirring in a 200 mL flask with a stopper. In a 100 mL glass beaker, 10.6 g DuroTak 87-4098 solution (polyacrylate adhesive with 38.5% adhesive solids), 250 mg isopropylmyristate, 250 mg lauryl alcohol, and 150 mg vardenafil (base, from LGM Pharma) were mixed with a magnetic stirrer. 5 g of the 10% Kollidon VA 64 solution in 2-propanol was added. The mixture was stirred for 60 minutes until homogenous. The resulting wet mix was coated onto sheets of release liner Scotchpak® 1022 using a Quadruple Film Applicator (Erichson Model 360) equipped with a glass plate to yield individual sheets of matrix-coated release liner. The coated sheets were transferred to a drying oven and kept for 20 minutes at 70° C. Thereafter, the sheets were removed from the oven, allowed to adjust to ambient temperature, and laminated with Dow™ BLF 2050 backing material to yield a three layer laminate. The three layer laminate was die cut into individual circular patches of 25 mm in diameter and individually packaged in heat sealable aluminum laminate sachets.

The resulting crystal free formulation was subjected to an in vitro-permeation test through Heat Separated Human Epidermis (HSHE), which is highly predictive for in vivo-transdermal flux rates.

Permeation experiments were conducted following a Standard Operating Procedure for using Hanson Vertical Diffusion Cells (7 mL) mounted with Heat Separated Human Epidermis, an in vitro-experiment with high predictability for transdermal in vivo-fluxes. A circular patch of 25 mm in diameter was, after removal of the release liner, applied on Heat Separated Human Epidermis. 0.5% Cavamax™ in PBS buffer served as the acceptor medium for the permeation experiments. Samples were withdrawn at predetermined time intervals (see Table 3 below) and subjected to HPLC quantification of vardenafil (V).

TABLE 3
Results of Permeation Experiments with Formulation 2 for Cell 1

| Time | Cumulative amount permeated V |
|---|---|
| (h) | (μg/cm²) |
| 0 | Below lower limit of detection |
| 4 | 0.22206 |
| 8 | 0.63642 |
| 12 | 1.14680 |
| 16 | 1.70814 |
| 24 | 2.34007 |
| 48 | 3.15175 |

### Example 3

### Formulation 3

In a 10 mL flask with a stopper, 50 mg of vardenafil (base, from LGM Pharma), 50 mg testosterone (from Sigma Aldrich), 1 mL of stock solution of 2.5 g isopropylmyristate in 50 mL ethanol/water (70% v/v), and 100 mg copovidone were mixed with 3.9 g of ethanol/water (70% v/v) and shaken for 10 minutes at ambient temperature to obtain the formulation below. The resulting crystal free formulation was subjected to an in vitro-permeation test through Heat Separated Human Epidermis (HSHE), which is highly predictive for in vivo-transdermal flux rates.

### Formulation 3

1% vardenafil
1% testosterone
1% isopropylmyristate
2% copovidone
95% ethanol/water (70% v/v)

Permeation experiments were conducted in duplicate (using 2 cells) following a Standard Operating Procedure for using Hanson Vertical Diffusion Cells (7 mL) mounted with Heat Separated Human Epidermis, an in vitro-experiment with high predictability for transdermal in vivo-fluxes. 200 mg of the formulation were applied on each Heat Separated Human Epidermis. 0.5% Cavamax™ in PBS buffer served as the acceptor medium for the permeation experiments. Samples were withdrawn at predetermined time intervals (see Tables 4 and 5 below) and subjected to HPLC quantification of both vardenafil (V) and testosterone (T).

TABLE 4
Results of Permeation Experiments with Formulation 3 for Cell 1

| Time | Cumulative amount permeated V | Cumulative amount permeated T |
|---|---|---|
| (h) | (μg/cm²) | (μg/cm²) |
| 0 | Below lower limit of detection | Below lower limit of detection |
| 2 | Below lower limit of detection | 1.3794 |
| 4 | Below lower limit of detection | 4.5317 |
| 6 | Below lower limit of detection | 8.6953 |
| 8 | 6.4930 | 13.2788 |
| 12 | 15.0437 | 18.6921 |
| 18 | 22.1804 | 23.5761 |
| 24 | 28.5040 | 28.2354 |

TABLE 5
Results of Permeation Experiments with Formulation 3 for Cell 2

| Time | Cumulative amount permeated V | Cumulative amount permeated T |
|---|---|---|
| (h) | (μg/cm²) | (μg/cm²) |
| 0 | Below lower limit of detection | Below lower limit of detection |
| 2 | Below lower limit of detection | 1.0042 |
| 4 | Below lower limit of detection | 4.1553 |
| 6 | Below lower limit of detection | 6.2970 |
| 8 | Below lower limit of detection | 11.4066 |
| 12 | 7.9791 | 17.7077 |
| 18 | 18.0098 | 24.5265 |
| 24 | 24.9135 | 28.2554 |

As can be seen from the tables above, the average dose of testosterone cumulatively permeated through HSHE from Formulation 3 over 24 hours was 28 μg/cm², and the average dose of vardenafil cumulatively permeated through HSHE from Formulation 3 over 24 hours was 27 μg/cm² in vitro. However, in vivo, the typical application area is much larger than 1 cm². Rather, the typical application area is approximately 200 cm² (e g, skin of the torso, upper arms, etc.) for 5 grams of a liquid/gel-type dosage form. Therefore, an in vivo-administration of 5 g of Formulation 3 will deliver approximately 5.6 mg of testosterone and 5.4 mg of vardenafil per day systemically.

The documented systemic in vivo-delivery of testosterone from 5 g of a comparable marketed 1% testosterone-mono formulation (Androgel™) is approximately 5 mg hormone per day. This is further validation of the potential in vivo-delivery of both testosterone and vardenafil from Formulation 3, given that testosterone in Formulation 3 can be seen as an internal reference for vardenafil in the experiment. Furthermore, one of ordinary skill in the art would expect that a testosterone-free variant of Formulation 3 will deliver approximately the same daily vardenafil dose as the combination formulation tested and described above.

As such, this evidence demonstrates that it is feasible to deliver vardenafil alone or in combinations with testosterone via pharmaceutical administration systems transdermally in amounts needed to treat conditions like pulmonary hypertension (PH), pulmonary arterial hypertension (PAH), benign prostatic hyperplasia (BPH), erectile dysfunction (ED), male hypogonadism, and male disorders, among others.

### Example 4

### Formulation 4

In a 10 mL flask with a stopper, 250 mg of lauryl alcohol, 50 mg of vardenafil (base, from LGM Pharma), and 50 mg testosterone (from Sigma Aldrich) were mixed with 4.7 g of 1,2-propanediol and shaken for 10 minutes at ambient temperature to obtain the formulation below. The resulting crystal free formulation was subjected to an in vitro-permeation test through Heat Separated Human Epidermis (HSHE), which is highly predictive for in vivo-transdermal flux rates.

### Formulation 4

1% vardenafil
1% testosterone
5% lauryl alcohol
93% 1,2-propanediol

Permeation experiments were conducted in duplicate (using 2 cells) following a Standard Operating Procedure for using Hanson Vertical Diffusion Cells (7 mL) mounted with Heat Separated Human Epidermis, an in vitro-experiment with high predictability for transdermal in vivo-fluxes. 200 mg of the formulation were applied on each Heat Separated Human Epidermis. 0.5% Cavamax™ in PBS buffer served as the acceptor medium for the permeation experiments. Samples were withdrawn at predetermined time intervals (see Tables 6 and 7 below) and subjected to HPLC quantification of both vardenafil (V) and testosterone (T).

TABLE 6
Results of Permeation Experiments with Formulation 4 for Cell 1

| Time | Cumulative amount permeated V | Cumulative amount permeated T |
|---|---|---|
| (h) | (μg/cm²) | (μg/cm²) |
| 0 | 0.0000 | 0.0000 |
| 2 | 6.7217 | 9.2628 |
| 4 | 17.0411 | 19.1794 |
| 6 | 25.6617 | 25.0057 |
| 8 | 33.0847 | 33.1854 |
| 12 | 39.8237 | 41.8837 |
| 18 | 44.5968 | 54.5857 |
| 24 | 43.7579 | 65.5107 |

TABLE 7
Results of Permeation Experiments with Formulation 4 for Cell *

| Time | Cumulative amount permeated V | Cumulative amount permeated T |
|---|---|---|
| (h) | (μg/cm²) | (μg/cm²) |
| 0 | Below lower limit of detection | 0.0000 |
| 2 | Below lower limit of detection | 0.0000 |
| 4 | 10.1029 | 10.5534 |
| 6 | 17.0177 | 17.1390 |
| 8 | 19.6641 | 20.4159 |
| 12 | 22.7994 | 26.3270 |
| 18 | 25.5547 | 33.1986 |
| 24 | 28.5597 | 45.4199 |

### Example 5

### Formulation 5

In a 10 mL flask with a stopper, 250 mg lauric acid, 100 mg copovidone, 50 mg of vardenafil (base, from LGM Pharma), and 50 mg testosterone (from Sigma Aldrich) were mixed with 4.6 g of dimethyl isosorbide and shaken for 10 minutes at ambient temperature to obtain the formulation below. The resulting crystal free formulation was subjected to an in vitro-permeation test through Heat Separated Human Epidermis (HSHE), which is highly predictive for in vivo-transdermal flux rates.

### Formulation 5

1% vardenafil
1% testosterone
5% lauric acid
2% copovidone
91% dimethyl isosorbide

Permeation experiments were conducted in duplicate (using 2 cells) following a Standard Operating Procedure for using Hanson Vertical Diffusion Cells (7 mL) mounted with Heat Separated Human Epidermis, an in vitro-experiment with high predictability for transdermal in vivo-fluxes. 200 mg of the formulation were applied on each Heat Separated Human Epidermis. 0.5% Cavamax™ in PBS buffer served as the acceptor medium for the permeation experiments. Samples were withdrawn at predetermined time intervals (see Tables 8 and 9 below) and subjected to HPLC quantification of both vardenafil (V) and testosterone (T).

TABLE 8
Results of Permeation Experiments with Formulation 5 for Cell 1

| Time | Cumulative amount permeated V | Cumulative amount permeated T |
|---|---|---|
| (h) | (μg/cm²) | (μg/cm²) |
| 0 | Below lower limit of detection | Below lower limit of detection |
| 2 | Below lower limit of detection | Below lower limit of detection |
| 4 | Below lower limit of detection | Below lower limit of detection |
| 6 | Below lower limit of detection | Below lower limit of detection |
| 8 | Below lower limit of detection | Below lower limit of detection |
| 12 | Below lower limit of detection | 0.1589 |
| 18 | Below lower limit of detection | 0.4961 |
| 24 | Below lower limit of detection | 1.3798 |

TABLE 9
Results of Permeation Experiments with Formulation 5 for Cell 2

| Time | Cumulative amount permeated V | Cumulative amount permeated T |
|---|---|---|
| (h) | (μg/cm²) | (μg/cm²) |
| 0 | Below lower limit of detection | Below lower limit of detection |
| 2 | Below lower limit of detection | Below lower limit of detection |
| 4 | Below lower limit of detection | Below lower limit of detection |
| 6 | Below lower limit of detection | 0.3340 |
| 8 | Below lower limit of detection | 0.2461 |
| 12 | Below lower limit of detection | 0.1813 |
| 18 | Below lower limit of detection | 0.3575 |
| 24 | Below lower limit of detection | 1.2627 |

In Formulation 5, the recovery rate of vardenafil could not be determined given the transformation of vardenafil to a new compound, which partly overlaps in the HPLC spectra. Analytical data indicate that vardenafil in Formulation 5 was oxidized at the amine nitrogen of the piperazine. In the ESI MS (electrospray ionization mass spectroscopy) spectra, a molecular mass of 16 mass units higher, which is equal to the formal addition of one atom of oxygen to vardenafil, was detected. In 2003, Seidel et al. from Bayer AG described the oxidation of a labeled vardenafil for synthesis of potential metabolites of vardenafil (Seidel, D., et al., Synthesis of [¹⁴C]-labelled vardenafil hydrochloride and metabolites. Journal of Labelled Compounds and Radiopharmaceuticals, 2003. 46(11): p. 1019-1032.). This oxidation also took place at the amine nitrogen of the piperazine. Comparing Formulation 5 with Formulations 3 and 4 shows that the main difference is the presence of dimethyl isosorbide and lauric acid in Formulation 5. Measuring the pH of all three formulations revealed that Formulation 3 had a pH of 7 and Formulation 4 had a pH of 6. In contrast, Formulation 5 had a pH of 4, which likely lead to a protonation of the piperazine nitrogen of vardenafil in the formulation, potentially making it more prone to oxidation. Furthermore, it cannot be excluded that dimethylisosorbide, which has ether partial structure, might have been partially autoxidized (see D. E. Clark, Peroxides and Peroxide-Forming Compounds, 2000, at http://www.bnl.gov/esh/cms/pdf/peroxides.pdf) during storage prior to its use in Formulation 5. It thus may have contained hydroperoxides. Hydroperoxides are strong oxidizing agents. The potential presence of hydroperoxides in dimethylisosorbide could have contributed to vardenafil's degradation in Formulation 5 as well. Given the observed oxidative degradation of vardenafil in Formulation 5, it is reasonable to expect that stabilization of that formulation (e.g., via addition of an antioxidant) will result in a more stable system with a higher concentration of the active agent and correspondingly higher transdermal flux rates of vardenafil.

### Example 6

### Formulation 6

In a 10 mL flask with a stopper, 100 mg of vardenafil (base, from LGM Pharma), 100 mg testosterone (from Sigma Aldrich), 200 mg myristyl alcohol, 300 mg Kollidon VA 64, and 1.0 g of 1,2-propanediol were mixed with 8.2 g of ethanol/water (70% v/v). While agitating the mixture, 100 mg Klucel® M PHARM Kremer hydroxypropylcellulose was added. The mixture was then allowed to gel. The resulting crystal free formulation was subjected to an in vitro-permeation test through Heat Separated Human Epidermis (HSHE), which is highly predictive for in vivo-transdermal flux rates.

Permeation experiments were conducted in duplicate (using 2 cells) following a Standard Operating Procedure for using Hanson Vertical Diffusion Cells (7 mL) mounted with Heat Separated Human Epidermis, an in vitro-experiment with high predictability for transdermal in vivo-fluxes. 200 mg of the formulation were applied on each Heat Separated Human Epidermis. 0.5% Cavamax™ in PBS buffer served as the acceptor medium for the permeation experiments. Samples were withdrawn at predetermined time intervals (see Tables 10 and 11 below) and subjected to HPLC quantification of both vardenafil (V) and testosterone (T).

TABLE 10
Results of Permeation Experiments with Formulation 6 for Cell 1

| Time | Cumulative amount permeated V | Cumulative amount permeated T |
|---|---|---|
| (h) | (μg/cm²) | (μg/cm²) |
| 0 | Below lower limit of detection | Below lower limit of detection |
| 2 | Below lower limit of detection | 0.38698 |
| 4 | Below lower limit of detection | 1.67822 |
| 8 | 0.12833 | 3.79415 |
| 12 | 0.22289 | 5.67499 |
| 16 | 0.29257 | 7.10318 |
| 24 | 3.93032 | 17.33918 |

TABLE 11
Results of Permeation Experiments with Formulation 6 for Cell 2

| Time | Cumulative amount permeated V | Cumulative amount permeated T |
|---|---|---|
| (h) | (μg/cm²) | (μg/cm²) |
| 0 | Below lower limit of detection | Below lower limit of detection |
| 2 | Below lower limit of detection | Below lower limit of detection |
| 4 | Below lower limit of detection | Below lower limit of detection |
| 8 | Below lower limit of detection | Below lower limit of detection |
| 12 | Below lower limit of detection | Below lower limit of detection |
| 16 | Below lower limit of detection | Below lower limit of detection |
| 24 | 1.63234 | 3.84506 |

It has been found that the co-administration of vardenafil and testosterone through human skin utilizing the embodiments of the present invention surprisingly delivers high daily doses of vardenafil. Application of 5 g of Formulation 3 onto approximately 200 cm² skin will deliver approximately 5.4 mg (transdermal flux rate: 27 μg/cm² over 24 h) of vardenafil and 5.6 mg (transdermal flux rate: 28 μg/cm² over 24 h) of testosterone per day systemically. Thus, Formulation 3 is capable of delivering approximately 7 times more vardenafil than needed for the once daily treatment of erectile dysfunction of 0.75 mg (see Table 12). The high transdermal flux rate of vardenafil even allows for a significant reduction of the application area and dose applied, which is a clear patient benefit in the treatment of erectile dysfunction and other diseases. It also indicates that formulations without penetration enhancers should deliver sufficiently high systemic doses of vardenafil upon transdermal administration. This will likely reduce the skin irritation potential of the resulting formulations and thereby provide an additional patient benefit.

While there is no literature data relative to transdermal flux rates through human skin available for other PDE V inhibitors, the high flux rate of vardenafil from Formulation 3 shows that it is significantly more suitable than most other PDE V inhibitors (or erectile dysfunction drugs) for transdermal administration alone or co-administered with testosterone. This becomes particularly evident looking at the comparison of vardenafil with sildenafil. Table 12 shows that it would take approximately 14 times higher transdermal flux rates (which are unlikely to be achieved) or 14 times higher doses/larger application areas (which are less patient friendly) of sildenafil than of vardenafil to treat erectile dysfunction patients with a once daily transdermal regimen. Taken together, the current invention disclosing vardenafil's unexpectedly high flux rates through human skin has a clear advantage over the conventional art.

TABLE 12
Calculation of required transdermal flux rates of vardenafil and sildenafil for the
once daily treatment of erectile dysfunction (ED)

| | | | | | | | | calculated | |
|---|---|---|---|---|---|---|---|---|---|
| | | | | | | | | transdermal | |
| | | | | | | | | flux rates | |
| | | | | | | | systemically | required to | |
| | | | | | | | available | deliver | |
| | | | on | | systemically | once | required | systemic | |
| | | | demand | | available | daily | dose for | doses for | |
| | | | oral dose | | dose for | oral dose | once daily | treatment | transdermal |
| | | | for | | treatment of | for | treatment of | of ED upon | flux rate |
| | | log | treatment | oral | ED | treatment | ED | application | achieved |
| Drug | M rel | P | of ED | bioavailability | (calculated) | of ED | (calculated) | on 200 cm² | (formulation) |
| vardenafil | 488.6 | 1.4 | 2.5 mg, | 15% | 0.38 mg, | n.a. | 0.75 mg | 3.8 μg/cm² | 27 μg/cm² |
| | | | 5 mg, | | 0.75 mg, | | | over 24 h | over 24 h |
| | | | 10 mg, | | 1.5 mg, | | | | (Formulation |
| | | | 20 mg | | 3 mg | | | | 3) |
| sildenafil | 474.6 | 1.9 | 25 mg, | 41% | 10.3 mg, | n.a. | 10.3 mg | 52 μg/cm² | n.a. |
| | | | 50 mg, | | 20.5 mg, | | | over 24 h | |
| | | | 100 mg | | 41 mg | | | | |
| tadalafil | 389.4 | 1.7 | 5 mg, | not | n.a. | 2.5 mg, | n.a. | n.a. | n.a. |
| | | | 10 mg, | established | | 5 mg | | | |
| | | | 20 mg | | | (50% of | | | |
| | | | | | | oral on | | | |
| | | | | | | demand | | | |
| | | | | | | dose) | | | |

The transdermal flux rates of sildenafil needed to be approximately 14-fold higher than that of vardenafil to deliver equieffective doses of the drug. Thus, the surprisingly high flux data for vardenafil show its exceptional suitability for transdermal delivery, both alone and in combination with testosterone.

Table 13 provides a summary of the transdermal flux rates for Examples 1-4 and 6.

TABLE 13
Summary of transdermal flux data

| Example/Formulation | 1 | 2 | 3 | 4 | 6 |
|---|---|---|---|---|---|
| Flux of Vardenafil from Cell 1 (μg/cm² over 24 h) | 21.9 | 2.3 | 28.5 | 43.8 | 3.9 |
| Flux of Vardenafil from Cell 2 (Cell* for Formulation 4) | 14.7 | n.a. | 24.9 | 28.6 | 1.6 |
| (μg/cm² over 24 h) | | | | | |
| Average flux of Vardenafil (μg/cm² over 24 h) | 18.3 | 2.3 | 26.7 | 36.2 | 2.8 |
| Average Daily Vardenafil Dose Systemically Available | 3.7 | 0.5 | 5.3 | 7.2 | 0.6 |
| from an Application Area of 200 cm² (mg) | | | | | |
| Flux of Testosterone from Cell 1 (μg/cm² over 24 h) | n.a. | n.a. | 28.2 | 65.5 | 17.3 |
| Flux of Testosterone from Cell 2 (Cell* for Formulation 4) | n.a. | n.a. | 28.3 | 45.4 | 3.8 |
| (μg/cm² over 24 h) | | | | | |
| Average Flux of Testosterone (μg/cm² over 24 h) | n.a. | n.a. | 28.3 | 55.5 | 10.6 |
| Average Daily Testosterone Dose Systemically Available | n.a. | n.a. | 5.7 | 11.1 | 2.1 |
| from an Application Area of 200 cm² (mg) | | | | | |

### Example 7

### Vardenafil and Riociguat, Transdermal Gel Formulation

In 2 L glass beaker, 10 g vardenafil (base, LGM Pharma) and 15 g riociguat are dissolved in 960 g isopropanol/water (70% v/v) while stirring with a propeller mixer for 60 min at ambient temperature. 15 g hydroxyethyl cellulose (Natrosol™ 250, Ashland) are added over a period of 5 min, avoiding lump formation and the introduction of air into the mixture. The resulting mixture is stirred for an additional 2 h at ambient temperature.

Packaging variant A: 5 g aliquots of the gel are filled into heat sealable aluminum laminate sachets and heat sealed.

Packaging variant B: 100 g of the gel are filled into a metered pump dispenser.

Administration: Therapeutically effective amounts of the gel are applied to the skin of the patient (for example to the chest, shoulders, upper arm, or lower abdomen).

### Example 8

### Vardenafil and Iloprost, Transdermal Matrix Patch Formulation

In a 5 L glass beaker, 20 g vardenafil (base, LGM Pharma), 0.6 g iloprost (Tocris) and 1 g alpha tocopherol are added to 100 g of isopropanol under stirring. Subsequently, 2380.5 g (928.4 g solids) of the polyacrylate adhesive Duro-Tak 87-4287 (39% solids in ethyl acetate, Henkel) are added, and it is stirred for 1 h to yield a homogenous mixture. 50 g of isopropyl myristate are added, and it is stirred for an additional hour. The resulting wet mix is coated onto sheets of release liner Scotchpak® 1022 using a labcoater Coatmaster 510 (Erichsen) equipped with a glass plate and a 90 μm knife (Film Applicator System Wasag, Model 288) in a discontinuous process to yield individual sheets of matrix-coated release liner. The coated sheets are transferred to a drying oven and kept for 20 min at 70° C. Thereafter, the sheets are removed from the oven, allowed to adjust to ambient temperature and laminated with Dow™ BLF 2050 backing material to yield a three layer laminate.

Alternatively, the resulting wet mix can be coated onto the release liner using a continuous coating, drying and laminating equipment to yield the desired three layer laminate.

Packaging: Circular patches of 10 cm² in size are die cut from the laminate and individually packaged in heat sealable aluminum laminate sachets.

Administration: A therapeutically effective number of patches are applied, after removal of the release liner, onto the skin of the patient (for example to the chest, shoulders, upper arm, or lower abdomen).

### Example 9

### Vardenafil and Enclomifene, Transdermal Spray Formulation

In 2 L glass beaker, 5 g vardenafil hydrochloride and 20 g enclomifene are mixed with 920 g isopropanol/water (70% v/v) while stirring with a propeller mixer for 10 min at ambient temperature. 5 g bis(2-hydroypropyl)amine, 30 g myristyl alcohol, and 20 g polyvinyl pyrrolidon (Kollidon® 12 PF, BASF) are added, and the mixture is stirred for another 20 minutes at ambient temperature.

Packaging: 100 g of the mixture are filled into a metered pump dispenser.

Administration: Therapeutically effective amounts of the mixture are sprayed onto the skin of the patient (for example to the chest, shoulders, upper arm, or lower abdomen).

### Example 10

### Vardenafil and Ambrisentan, Transdermal Gel Formulation

In 2 L glass beaker, 5 g vardenafil (base, LGM Pharma) and 20 g ambrisentan are mixed with 755 g ethanol/water (70% v/v). 200 g 1,2 propanediol are added, and the resulting mixture is stirred with a propeller mixer for 60 min at ambient temperature. 20 g hydroxypropyl cellulose (Methocel™ MF, Ashland) are added over a period of 5 min, avoiding lump formation and the introduction of air into the mixture. The resulting mixture is stirred for an additional 2 h at ambient temperature.

Packaging variant A: 5 g aliquots of the gel are filled into heat sealable aluminum laminate sachets and heat sealed.

Packaging variant B: 100 g of the gel are filled into a metered pump dispenser.

Administration: Therapeutically effective amounts of the mixture are applied onto the skin of the patient (for example to the chest, shoulders, upper arm, or lower abdomen).

### Example 11

### Vardenafil, Transdermal Reservoir Patch Formulation

In 2 L glass beaker, 20 g vardenafil (base, LGM Pharma) are mixed with 760 g isopropanol/water (70% v/v) while stirring with a propeller mixer for 30 min at ambient temperature. 150 g 1,2-propanediol and 50 g lauryl alcohol are added, and the resulting mixture is stirred for another 60 minutes. 20 g hydroxyethyl cellulose (Natrosol™ 250, Ashland) are added over a period of 5 min, avoiding lump formation and the introduction of air into the mixture. The resulting mixture is stirred for an additional 2 h at ambient temperature.

2 g aliquots of the mixture are filled into the circular cavities of 3.5 cm in diameter of an impermeable film of polyethylene terephthalate. In a subsequent heat sealing operation, a separately manufactured three layer sheet material made of semipermeable ethylene vinyl acetate polymer film, a pressure sensitive adhesive coating of 40 μm thickness made from Dow Corning's Bio PSA 7-4302, and ScotchPak® 1022 release liner is heat sealed so that the ethylene vinyl acetate polymer film and the polyethylene terephthalate film form a circular bond surrounding the circular cavity. Patches of 5 cm in diameter are die cut from the resulting multi laminate so that the cavities lie in the center of the individual patch and a heat seal ring surrounds it.

Packaging: Individual patches are packaged in heat sealable aluminum laminate sachets.

Administration: One or more patches are, after removal of the release liner, applied onto the skin of the patient (for example to the chest, shoulders, upper arm, or lower abdomen).

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A pharmaceutical administration system for the transdermal application of at least one active agent, comprising:
vardenafil and/or a pharmaceutically acceptable salt thereof as the active agent; and
a pharmaceutically acceptable carrier providing a solution of the at least one active agent in the administration system, wherein the pharmaceutically acceptable carrier comprises at least one transdermal penetration enhancer;
wherein the system comprises an amount of penetration enhancer that is greater than an amount of vardenafil.

2. A pharmaceutical administration system for the transdermal application of at least one active agent, comprising:
vardenafil and/or a pharmaceutically acceptable salt thereof as the active agent;
testosterone as another active agent; and
a pharmaceutically acceptable carrier providing a solution of the at least one active agent in the administration system.

3. The system according to claim 1, wherein a content of vardenafil and/or a pharmaceutically acceptable salt thereof in the system is 0.1-15% by weight.

4. The system according to claim 2, wherein a content of testosterone in the system is 0.1-15% by weight.

5. The system according to claim 1, wherein the pharmaceutically acceptable carrier is an alcohol and/or a polyol or a mixture of alcohols and/or polyols.

6. The system according to claim 1, wherein the pharmaceutically acceptable carrier comprises a mixture of one or more alcohols and/or one or more polyols and water.

7. The system according to claim 1, wherein the pharmaceutically acceptable carrier comprises a gel forming agent.

8. The system according to claim 1, wherein the pharmaceutically acceptable carrier is a biocompatible pressure sensitive adhesive.

9. The system according to claim 1, wherein the pharmaceutically acceptable carrier is a mixture of biocompatible pressure sensitive adhesives.

10. (canceled)

11. The system according to claim 1, further comprising a second transdermal penetration enhancer.

12. The system according to claim 1, wherein the pharmaceutically acceptable carrier comprises a crystallization inhibitor.

13. A method of systemically delivering therapeutic doses of vardenafil and/or a pharmaceutically acceptable salt thereof, comprising:
applying the system according to claim 1 to the skin of a patient in need thereof.

14. A method of treating benign prostatic hyperplasia, comprising:
applying the system according to claim 1 to skin of a patient in need thereof.

15. A method of treating benign prostatic hyperplasia, comprising:
applying the system according to claim 2 to skin of a patient in need thereof.

16. A method of treating erectile dysfunction, comprising:
applying the system according to claim 1 to skin of a patient in need thereof.

17. A method of treating male hypogonadism, comprising:
applying the system according to claim 2 to skin of a patient in need thereof.

18. A method of treating male hypogonadism and erectile dysfunction simultaneously, comprising:
applying the system according to claim 2 to skin of a patient in need thereof.

19. A method of treating pulmonary arterial hypertension, comprising:
applying the system according to claim 1 to the skin of a patient in need thereof.

20. A method of treating pulmonary hypertension, comprising:
applying the system according to claim 1 to the skin of a patient in need thereof.

21. A method of treating either PH, PAH, ED, or BPH in patients who simultaneously suffer from a liver condition which contraindicates oral PDE V inhibitor administration, comprising:
applying the system according to claim 1 to the skin of a patient in need thereof.

22. The system according to claim 2, wherein the system achieves continuous transdermal delivery of vardenafil over a time period of at least 12 hours.

23. A pharmaceutical administration system for the transdermal application of at least one active agent, comprising:
vardenafil and/or a pharmaceutically acceptable salt thereof as the active agent; and
a pharmaceutically acceptable carrier providing a solution of the at least one active agent in the administration system, wherein the pharmaceutically acceptable carrier comprises at least one crystallization inhibitor selected from the group consisting of poly vinyl pyrrolidone, vinylpyrrolidone vinyl acetate copolymer, colloidal silicon dioxide, and hydrophobic silicon dioxides.

24. A pharmaceutical administration system for the transdermal application of at least one active agent, comprising:
vardenafil and/or a pharmaceutically acceptable salt thereof as the active agent; and
a pharmaceutically acceptable carrier providing a solution of the at least one active agent in the administration system; and
wherein the system is devoid of water as a solvent.
